(19) United States
(12) Patent Application Publication
(10) Pub. No.: US 2006/0046278 A1
(43) Pub. Date: Mar. 2, 2006

(54) Methods and kits for determining metabolic stability of compounds

(75) Inventors: John H. Ansede, Cary, NC (US); Dhiren R. Thakker, Raleigh, NC (US)

Correspondence Address: MYERS BIGEL SIBLEY & SAJOVEC, PO BOX 37428, RALEIGH, NC, 27627, US

(73) Assignee: Qualyst, Inc.

(21) Appl. No.: 10/926,500

(22) Filed: Aug. 26, 2004

(51) Int. Cl. C12Q 1/26; C12N 9/02

(52) U.S. Cl. 435/25; 435/189

(57) ABSTRACT

The present invention provides methods and kits for determining the metabolic stability of compounds (e.g., stability to oxidative metabolism). In particular embodiments, the invention provides a method of determining the susceptibility of a compound to metabolism by an enzyme. In one representative embodiment, the enzyme is a cytochrome P450 enzyme. The invention is well-adapted for use in high throughput drug screening programs.

### FIELD OF THE INVENTION

The present invention pertains to methods and kits for of assessing the metabolic stability of compounds; in particular, the present invention relates to methods and kits for determining the susceptibility of a compound to metabolism by an enzyme.

### BACKGROUND OF THE INVENTION

The drug discovery process has evolved over the past sixty years from serendipitous findings of biologically active natural products to rational design of potent and selective pharmacologically active compounds based on elucidation of three-dimensional structure of target proteins (Burkhard et al., (1999) J. Mol. Biol. 287:853; Shuker et al., (1996) Science 274:1531; Kiyama et al., (1999) J. Med. Chem. 42: 1723), to high-throughput screening against cloned and expressed enzymes and receptors (Broach et al., (1996) Nature 384:14; Fernandes, (1998) Curr. Opin. Chem. Biol. 2: 597; Silverman et al., (1998) Curr. Opin. Chem. Biol. 2:397), to the construction of enormously diverse combinatorial libraries (Gordon et al., (1994) J. Med. Chem. 37:1385; Gallop et al., (1994) J. Med. Chem. 37:1233; Fecik et al., (1998) Med. Res. Rev. 18:149) for ultra high-throughput screening. Progress in high-throughput screening and robotics (Persidis, (1998) Nat. Biotechnol. 16:488; Houston, (1997) Methods Find Exp. Clin. Pharmacol. 19:43; Houston et al., (1997) Curr. Opin. Biotechnol. 8:734) has made it possible to screen chemical libraries for biological activity at rates in excess of 50,000-100,000 compounds per week. The sequencing of the human genome has led to the identification of greater than 30,000 genes (Venter et al., (2001) Science 291:1304; Lander et al., (2001) Nature 409:860) of which 1,000 may be implicated in the emergence or cause of a disease, yielding up to 5,000 to 10,000 potential targets for drug therapy (Drews, (2000) Science 287:1960), although the advances in genome science have yet to translate into improved human therapeutics. Overall, major advances in chemistry, molecular biology and high-throughput technology have provided discovery programs the necessary means for screening enormous number of compounds against a large number of targets to identify leads in a relatively short time, often less than a year. These leads then undergo more vigorous tests to identify those compounds with optimal “drug-like” properties (i.e., adequate physico-chemical stability, solubility, safety, efficacy, in vivo disposition).

Thus, despite rapid and efficient identification of leads, it takes several years, in some cases up to fifteen years, to bring a drug from discovery to market with a most recently estimated price in excess of $800 million (US) per individual drug (DiMasi et al., (2003) J. Health Econ. 22:151). These high costs cannot be solely attributed to inflation or extensive clinical testing required by federal agencies; they also reflect the high rate of failure in the preclinical and clinical development of drugs. The high cost of bringing a new drug therapy to market is juxtaposed against the swelling public opinion to bring the prices of prescription medicines down, placing tremendous pressure on pharmaceutical companies to reduce time-to-market and decrease discovery/development costs. Hence, major efforts are directed toward identifying and eliminating compounds (or compound classes) that are not likely to have “drug-like” properties at earlier stages of discovery.

The three main reasons a drug fails during clinical trials are lack of efficacy, unacceptable adverse effects, and unfavorable ADME properties. Therefore the ultimate success of a compound is not only defined by its biological activity and potency, but also by its ADME/toxicity properties. Although high-throughput screening has substantially increased the number of lead compounds, most of these compounds are eliminated during additional screening and testing. Of the drug candidates that enter clinical development phase, over 40% fail to make it to the market due to unfavorable drug metabolism and pharmacokinetic properties, with an additional 11% eliminated due to toxicity. Hence, lead optimization programs have incorporated screens to select drugs with desirable ADME/toxicity properties to enhance the prospect that these new lead compounds will have higher success rates as they make their way to the clinic. While advances in high-throughput technology has allowed for a dramatic increase in the number of lead compounds, technology used in developing screens for pharmacokinetic properties have lagged behind causing a bottleneck in the drug discovery process. For example, high-throughput assays used in screening combinatorial libraries can achieve rates in excess of 50,000-100,000 compounds per week; however the fastest methods currently in use for ADME screening are several orders of magnitude slower (White, (2000) Annu Rev. Pharmacol. Toxicol. 40:133). High-throughput rates are not essential for screening compounds that have already been selected as lead candidates for optimal ADME properties, because this compound pool is much smaller. However, several leading pharmaceutical companies have made a strategic decision to implement high-throughput assays to screen their chemical libraries for compounds with acceptable ADME properties in parallel to selecting compounds with potent biological activity against a therapeutic target.

Metabolic transformation of drug molecules represents a key process by which drugs are cleared from the body. Metabolic transformations have traditionally been divided into two phases. Phase I reactions (biotransformation) include oxidation, reduction, and hydrolysis which primarily serve to increase the hydrophilicity and enhance the excretion of a drug by unveiling or incorporating a polar functional group into the molecule (OH, SH, NH₂, or CO₂H). Phase II reactions (conjugation) further increase the polarity of a drug by modifying a functional group to form O— or N— glucuronides, sulfate esters, α-carboxyamides and glutathionyl adducts. Metabolic stability is one of several major determinates in defining the oral bioavailability and systemic clearance of a drug. After a drug is administered orally, it first encounters metabolic enzymes in the gastrointestinal lumen as well as in the intestinal epithelium. After it is absorbed into the bloodstream through the intestinal epithelium, it is first delivered to the liver via the portal vein. A drug can be effectively cleared by intestinal or hepatic metabolism before it reaches systemic circulation, a process known as first pass metabolism. The stability of a compound towards metabolism within the liver as well as extrahepatic tissues will ultimately determine the concentration of drug found in the systemic circulation and affect its half-life and residence time within the body.

Oxidative metabolism is the most common biotransformation reaction in drug metabolism catalyzed by a superfamily of membrane-bound mixed-function oxidases, known as the cytochrome P450 (CYP) monooxygenase system, located in the smooth endoplasmic reticulum of the liver and other extraheptic tissues. The functional enzyme system consists of two components, the heme-protein CYP and the flavoprotein NADPH-CYP reductase. The active site of CYP consists of a hydrophobic substrate-binding domain in which is embedded an iron protoporphyrin prosthetic group axially coordinated to a cysteine residue of the apoprotein. Substrate and dioxygen binding as well as electron transfer occurs in a coordinated fashion to produce an activated oxygen molecule that results in the insertion of one oxygen atom into the substrate, concomitant to the production of a water molecule. The function of NADPH-CYP reductase is to transfer electrons from NADPH to the CYP active site. The catalytic cycle of CYP has been the focus of numerous reviews (Guengerich, (2001) Chem Res. Toxicol. 14:611; Porter et al., (1991) J. Biol. Chem. 266:13469; White et al., Annu Rev. Biochem 49:315; Anzenbacher et al., (2001) Cell Mol. Life Sci. 58:737).

The human body has a unique challenge in having to metabolize a vast array of lipophilic compounds, which makes it impractical to synthesize one enzyme with a specific active site for each compound. Therefore, to overcome this problem, CYPs have broad substrate specificities and regioselectivities. Furthermore, multiple CYP isoforms are expressed in the liver and extrahepatic tissues that have differential specificities for various compounds, yet at the same time a compound may be metabolized by multiple CYP isoforms. While the liver is the main site of CYP-catalyzed oxidative metabolism for orally administered drugs (White et al., (1980) Annu Rev Biochem 49:315 and references therein), high levels of CYP isoforms have also been found in extrahepatic tissues including the intestinal epithelium, kidney, lung, and nervous tissue. There are eleven major drug-metabolizing CYPs that are expressed in the liver, including CYP1A2, CYP2A6, CYP2B6, CYP2C8/9/18/19, CYP2D6, CYP2E1, and CYP3A4/5. Among these isoforms, CYP1A2, CYP2C9, CYP2C19, CYP2D6 and CYP3A4 are responsible for the metabolism of over 90% of all drugs (Rendic et al., (1997) Drug Metab. Rev. 29:413 and references therein). The most abundantly expressed CYP isoform is CYP3A4, and is responsible for the metabolism of greater then 50% of pharmaceuticals (Guengerich, (1992) FASEB J. 6:745; Guengerich, (1996) J. Pharmacokinet. Biopharm. 24:521). CYP3A4 exhibits a high degree of inter- and intra-individual variability (Shimada et al., (1994) J. Pharmacol. Exp. Ther. 270:414), which can make it difficult to achieve a certain range of plasma concentrations in all patients if a drug is predominantly or exclusively cleared by this enzyme. Furthermore, genetic polymorphism (large differences in expression of a protein among two or more patient populations due to multiple alleles) among CYP isoforms, such as CYP2D6, can cause large variations in plasma concentrations of certain drugs, and further complicate the ability to predict how patient population will react to these drugs. A drug cleared exclusively by such an enzyme requires careful consideration of dosing adjustments according to the patient populations in order to avoid adverse effects due to overdose. Clearly, stability of drug candidates to metabolism by CYP isoforms can define their therapeutic and adverse effect profiles in many cases. Hence, it is important to evaluate the metabolic stability of drug candidates towards CYP-mediated oxidative metabolism, and to use it as a parameter in identifying and optimizing drug candidates for clinical development.

From the foregoing discussion, it is apparent that methods, kits and reagents for determining the metabolism of a drug or other foreign compounds by enzymes involved in biotransformation, in particular CYP, have implications for drug development.

### SUMMARY OF THE INVENTION

The present invention provides methods, reagents and kits for determining the metabolic stability of compounds (e.g., stability to oxidative metabolism). In addition, the invention is well-adapted for use in high throughput drug screening programs.

Accordingly, as a first aspect, the invention provides a method of determining the susceptibility of a compound to metabolism by an enzyme, the method comprising: (a) contacting (e.g., incubating) a compound with an enzyme to produce a reaction product mixture, wherein metabolism of the compound by the enzyme produces a reactive oxygen species as a side product in the reaction product mixture; then (b) contacting (e.g., allowing to react) at least some of the reaction product mixture of (a) with a detection reagent comprising an indicator compound precursor, wherein reaction between reactive oxygen species and the detection reagent results in the production of an indicator compound from the indicator compound precursor; and (c) detecting the presence or absence of the indicator compound, wherein the presence of the indicator compound indicates the susceptibility of the compound to metabolism by the enzyme, thereby determining the susceptibility of a compound to metabolism by the enzyme. According to this particular embodiment, the enzyme reaction of (a) and the detection reaction of (b) are carried out in two or more separate reaction steps.

As a second aspect, the invention provides a method of determining the susceptibility of a compound to metabolism by an enzyme, the method comprising: (a) contacting (e.g., incubating) a compound with an enzyme to produce a reaction product mixture, wherein metabolism of the compound by the enzyme produces a reactive oxygen species as a side product in the reaction product mixture; (b) removing at least some of the reaction product mixture of (a) and contacting (e.g., allowing to react) with a detection reagent comprising an indicator compound precursor, wherein reaction between reactive oxygen species in the reaction product mixture and the detection reagent results in the production of an indicator compound from the indicator compound precursor; and (c) detecting the presence or absence of the indicator compound, wherein the presence of the indicator compound indicates the susceptibility of the compound to metabolism by the enzyme, thereby determining the susceptibility of a compound to metabolism by the enzyme.

In particular embodiments of the invention, the enzyme is a cytochrome P450 (CYP) enzyme.

As yet another aspect, the invention provides a method of determining the susceptibility of a compound to metabolism by a CYP enzyme, the method comprising: (a) contacting (e.g., incubating) a compound with a CYP enzyme to produce a reaction product mixture, wherein metabolism of the compound by the CYP enzyme produces a reactive oxygen species as a side product in the reaction product mixture; (b) contacting (e.g., allowing to react) all or a portion of the reaction product mixture of (a) with a detection reagent comprising a chemiluminescent compound precursor and trichlorophenyloxalate (TCPO), wherein reaction between reactive oxygen species in the reaction product mixture and the detection reagent results in the production of a chemiluminescent compound from the chemiluminescent compound precursor; and (c) detecting the presence or absence of the chemiluminescent compound, wherein the presence of the chemiluminescent compound indicates the susceptibility of the compound to metabolism by the cytochrome P450 enzyme, thereby determining the susceptibility of a compound to metabolism by the cytochrome P450 enzyme.

Also provided are kits for practicing the methods of the invention, the kits comprising (a) an enzyme; (b) trichlorophenyloxalate (TCPO); and (c) a chemiluminescent compound precursor.

These and other aspects of the invention are set forth in more detail in the description of the invention below.

### BRIEF DESCRIPTION OF THE DRAWINGS

FIG. 1. Illustration of a trichlorophenyloxalate (TCPO) based peroxyoxalate chemiluminescence reaction scheme.

FIG. 2. The production of ROS as measured by chemiluminescence (CL) generation during CYP2D6 oxidative metabolism of dextromethorphan (solid squares) and desipramine (solid circles). Substrates were tested at 20 μM as described in the Examples (Section 1.2.1).

FIG. 3. The metabolic stability towards CYP2D6 oxidative metabolism as measured by parent compound loss using LC-MS detection for 11 test compounds. Compounds were tested at 5 μM final concentration and the amount of parent compound remaining in the reactions after 10 min of incubation is shown.

FIG. 4. The effect of substrate on chemiluminescence production in CYP2D6 reactions.

### DETAILED DESCRIPTION OF THE INVENTION

There is a need in the art for improved methods of determining the metabolic stability of compounds, in particular, the stability of compounds that are candidates for use as therapeutic agents for veterinary and/or medical purposes to enzymatic metabolism. For example, as part of the drug screening and identification process, it is useful to determine the susceptibility of a candidate compound (e.g., a compound that is of possible interest for a particular therapeutic application) to metabolism by enzymes such as enzymes that are involved in the biotransformation of xenobiotics, including cytochrome P450 (CYP) enzymes. The present invention provides methods and kits for determining the susceptibility of a compound to an enzyme(s) of interest, including enzymes that are involved in biotransformation processes such as CYP enzymes. Further, the instant invention is amenable to high throughout screening methods in which a plurality (e.g., two or more) compounds can be assessed in parallel for susceptibility to metabolism by a selected enzyme(s).

One advantage of the methods of the present invention is that they do not require direct measurement of either the compound or its metabolite. Thus, the invention provides universal methods that can be applied to evaluate the metabolism of compounds without the requirement to directly detect the compound or its metabolites and can be applied to the evaluation of new and/or uncharacterized compounds, and are particularly well-adapted for high throughput formats.

The present invention will now be described with reference to the accompanying drawings, in which preferred embodiments of the invention are shown. This invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

As used in the description of the invention and the appended claims, the singular forms “a,” “an” and “the” are intended to include the plural forms as well, unless the context clearly indicates otherwise.

As one aspect, the invention provides a method of determining the susceptibility of a compound to metabolism by an enzyme, the method comprising: (a) contacting a compound with an enzyme to produce a reaction product mixture, wherein metabolism of the compound by the enzyme produces a reactive oxygen species (“ROS”) as a side product in the reaction product mixture, and then (b) contacting at least some of the reaction product mixture of (a) with a detection reagent comprising an indicator compound precursor, wherein reaction between ROS in the reaction product mixture and the detection reagent results in the production of an indicator compound from the indicator compound precursor; and (c) detecting the presence or absence of the indicator compound, wherein the presence of the indicator compound indicates the susceptibility of the compound to metabolism by the enzyme. According to this particular embodiment, the enzyme reaction of (a) and the detection reaction of (b) are carried out in two or more separate reaction steps. The enzyme and detection reactions can be carried out in the same or separate reaction vessels (e.g., beaker, flask, test tube, microfuge tube, multiwell plate, chromatography column, culture dish, and the like), and there can be intermediate steps between the enzyme reaction and the detection reaction. Further, it is not necessary that the enzyme reaction be taken to completion prior to initiation of the detection reaction.

To illustrate, in one exemplary embodiment, a compound is contacted with an enzyme under conditions suitable for activity of the enzyme, wherein metabolism of the compound by the enzyme produces a ROS as a side product in a side reaction. The enzyme reaction is allowed to continue for the desired time and then the detection reagent is added to the reaction mixture (i.e., in the same reaction vessel) to start the detection reaction whereby the indicator compound precursor is converted to the indicator compound and a detectable signal is generated that indicates the susceptibility of the compound to metabolism by the enzyme.

Alternatively, the invention can be carried out by allowing the enzyme reaction to proceed for a desired period of time, and then removing at least some of the reaction product mixture and contacting the reaction product mixture with the detection reagent. In representative embodiments, the method comprises (a) contacting a compound with an enzyme to produce a reaction product mixture, wherein metabolism of the compound by the enzyme produces a ROS as a side product in the reaction product mixture; (b) removing at least some of the reaction product mixture of (a) and contacting with a detection reagent comprising an indicator compound precursor, wherein reaction between ROS in the reaction product mixture and the detection reagent results in the production of an indicator compound from the indicator compound precursor; and (c) detecting the presence or absence of the indicator compound, wherein the presence of the indicator compound indicates the susceptibility of the compound to metabolism by the enzyme. Optionally, all or a portion of the enzyme reaction product mixture of (a) is removed and is added to a separate reaction vessel to carry out the detection reaction of (b). In representative methods, aliquots of the enzyme product reaction mixture of (a) are removed at multiple (e.g., two, three, four, five, six or more) time points after initiation of the reaction and contacted with the detection reagent so as to follow metabolism of the compound over time. For example, according to this embodiment, the metabolism of the compound can be determined at multiple time points to determine a rate of metabolism of the compound. As described above, there can be one or more intermediate steps between the enzyme and detection reactions.

By carrying out the enzymatic and detection reactions as separate reactions (i.e., temporally, and optionally spatially, separated), the present invention permits a wider range of reaction conditions for each reaction. For example, some components of the detection reagent may not be compatible with the enzyme reaction (e.g., they may inactivate the enzyme). As another consideration, some components of the enzyme reaction may interfere with the detection reaction and/or detection of the indicator compound. As another illustration, according to the present invention the enzyme reaction and detection reaction can be carried out in separate phases. For example, the enzyme reaction can be carried out in an aqueous phase and the detection reaction carried out in a nonaqueous (i.e., nonpolar, organic) comprising, consisting essentially of or consisting of a suitable nonaqueous solvent such as ethyl acetate, methylene chloride, acetic acid, acetone, acetonitrile, aniline; benzene, benzonitrile, benzyl alcohol, bromobenzene, bromoform, 1-butanol, 2-butanol, carbon disulfide, carbon tetrachloride, chlorobenzene, chloroform, cyclohexane, cyclohexanol, decalin, dibromethane, diethylene glycol, diethylene glycol ethers, diethyl ether, diglyme, dimethoxymethane, N,N-dimethylformamide, ethanol, ethylamine, ethylbenzene, ethylene glycol ethers, ethylene glycol, ethylene oxide, formaldehyde, formic acid, glycerol, heptane, hexane, iodobenzene, mesitylene, methanol, methoxybenzene, methylamine, methylene bromide, methylene chloride, methylpyridine, morpholine, naphthalene, nitrobenzene, nitromethane, octane, pentane, pentyl alcohol, phenol, 1-propanol, 2-propanol, pyridine, pyrrole, pyrrolidine, quinoline, 1,1,2,2-tetrachloroethane, tetrachloroethylene, tetrahydrofuran, tetrahydropyran, tetralin, tetramethylethylenediamine, thiophene, toluene, 1,2,4-trichlorobenzene, 1,1,1-trichloroethane, 1,1,2-trichloroethane, trichloroethylene, triethylamine, triethylene glycol dimethyl ether, 1,3,5-trimethylbenzene, m-xylene, o-xylene, p-xylene, 1,2-dichlorobenzene, 1,3-dichlorobenzene, 1,4-dichlorobenzene, and combinations thereof. Many enzymes exhibit substantial or complete activity loss under nonaqueous conditions. The methods of the present invention accommodate the enzyme and detection reactions being carried out in different phases by separating the two reactions.

The term “aqueous” phase does not exclude the presence of low concentrations (e.g., typically less than 1%) of nonaqueous components as long as the presence of the nonaqueous components does not substantially impair the activity of the enzyme.

The term “nonaqueous” phase refers to an organic or nonpolar phase but is not limited to anhydrous solvents. In embodiments of the invention, the nonaqueous phase comprises about 50% or more of nonpolar organic solvent. In particular embodiments, the nonaqueous phase comprises from about 50% to 100% of nonpolar organic solvent, about 0 to 30% water, and about 0 to 30% polar organic solvent. The nonaqueous phase can exist as a single phase or as an emulsion of two phases.

In representative embodiments, the enzyme reaction is carried out in an aqueous phase and some or all of the enzyme product reaction mixture is added to a nonaqueous detection reaction mixture. In some embodiments, all or a portion of the enzyme product reaction mixture is added to the detection reaction to achieve a final concentration of about 0.1 to about 7, 10, 12, 15, 20 or 25% (v/v).

As another aspect, the invention provides a method of determining the susceptibility of a compound to metabolism by a CYP enzyme, the method comprising (a) contacting a compound with a CYP enzyme to produce a reaction product mixture, wherein metabolism of the compound by the CYP enzyme produces a ROS as a side product; (b) contacting all or a portion of the reaction product mixture of (a) with a detection reagent comprising a chemiluminescent compound precursor and trichlorophenyloxalate (TCPO), wherein reaction between ROS in the reaction product mixture and the detection reagent results in the production of a chemiluminescent compound from the chemiluminescent compound precursor; and (c) detecting the presence or absence of the chemiluminescent compound, wherein the presence of the chemiluminescent compound indicates the susceptibility of the compound to metabolism by the CYP enzyme. According to this embodiment of the invention, the enzyme and detection steps of (a) and (b), respectively, can be carried out in the same or separate reactions. If carried out as separate reactions, the reactions of (a) and (b) can be performed in the same or different vessels as discussed above with respect to the preceding methods of the invention. For example, the detection reagent can be added to the vessel containing the enzyme reaction product mixture. In other representative embodiments, all or a portion of the enzyme reaction product mixture is removed and is added to a separate reaction vessel to carry out the detection reaction of (b). As discussed above, there can be one or more intermediate steps between the enzyme and detection reactions. Further, as described above with respect to the preceding methods of the invention, the enzyme reaction and detection reaction can be carried out in separate phases. For example, the enzyme reaction of (a) can be carried out in an aqueous phase and the detection reaction of (b) carried out in a nonaqueous phase.

Aliquots of the enzyme reaction product mixture of (a) can be removed at multiple (e.g., two, three, four, five, six or more) time points after initiation of the reaction and contacted with the detection reagent so as to follow metabolism of the compound over time, also as described above with respect to other methods of the invention (for example, to determine a rate of metabolism of the compound by the enzyme).

The methods of the invention can be qualitative or quantitative in nature. Further, the inventive methods can be carried out on one or a plurality (e.g., two or more compounds) at a time in parallel. For example, the method can be carried out in parallel on a plurality of compounds in a multi-well plate, e.g., in a 96-well, 384-well or 1536-well plate format.

The compound can be contacted with the enzyme and the enzyme reaction product mixture can be contacted with the detection reagent by any method known in the art. For example, the compound and the enzyme can be mixed together and/or the enzyme reaction product mixture and the detection reagent can be mixed together. For example, the enzyme and detection reactions can be carried out in a liquid state, and the compound and enzyme and/or the enzyme reaction product mixture and the detection reagent can be mixed together. Alternatively, some of the components can be affixed (e.g., covalently or non-covalently) to a solid support (including gel supports). For example, the compound, the enzyme and/or the indicator compound precursor can be affixed to a solid support. To illustrate, the compound, the enzyme and/or the indicator compound precursor can be affixed to the surface of a multi-well plate, a bead, a glass or plastic slide, a culture dish, a column matrix, and the like. In representative embodiments, the compound and/or the enzyme and/or the indicator compound precursor can be affixed to a multi-well plate and the other reagents added thereto. As a further illustration, the compound and/or the enzyme and/or the indicator compound precursor can be affixed to a bead or a column matrix.

Methods of affixing compounds covalently or non-covalently to solid supports, e.g., by drying, electrostatic interactions, chemical and/or affinity conjugation are well known in the art.

The methods of the invention can be carried out to determine the susceptibility of a compound to metabolism by a selected enzyme (i.e., an enzyme of interest for evaluation). The compound is contacted with the enzyme under conditions sufficient for enzyme activity, e.g., in the presence of any cofactors, buffers, salts, detergents and/or other reagents that are necessary for enzyme activity, at a pH and temperature that are suitable for enzyme activity, and the like. The enzyme has a primary metabolic activity (i.e., which results in the metabolism of the compound) and a secondary or a side reaction associated with the primary metabolic activity of the enzyme. The side reaction produces a chemical side product, which is a reactive oxygen species (ROS).

The enzyme can be an enzyme that is involved in the biotransformation of xenobiotics. An illustrative example of an enzyme includes but is not limited to CYP enzymes.

The inventive methods can be carried out with one enzyme or with two or more enzymes in the enzyme reaction mixture.

The terms “cytochrome P450” enzyme and “CYP” enzyme refer to a large family (often called a “superfamily”) of enzymes. As used herein, these terms are meant to encompass all members of the CYP superfamily and include CYP enzymes of microbial (e.g., bacterial or yeast), fungal, plant, invertebrate (e.g., insect) and vertebrate (e.g., mammalian including human, simian, bovine, porcine, ovine, caprine, canine, feline, equine, mouse, rat, rabbit and the like) origin as well as enzymes that are purified from natural sources, are wholly or partially synthetic, or are produced using recombinant nucleic acid methods. Also encompassed are modified CYP enzymes (e.g., fusion proteins or enzymes modified by mutation such as deletion, insertion, substitution or truncation to alter the properties of the enzyme including but not limited to enzymatic activity, stability and the like or to facilitate purification, conjugation and/or detection of the enzyme).

All isoenzymes, or isoforms, within the CYP superfamily are contemplated to fall within the terms “cytochrome P450” enzyme and “CYP” enzyme as used herein. In particular embodiments, the CYP enzyme includes CYP1A2, CYP2A6, CYP2B6, CYP2C8, CYP2C9, CYP2C10, CYP2C18, CYP2C19, CYP2D6, CYP2E (including CYP2E1), CYP3A4, CYP3A5 and/or CYP3A7 isoforms as these isoforms have been identified as those most commonly responsible for the metabolism of drugs in humans. Additional CYP superfamily members are described in U.S. Pat. Nos. 5,786,191, 5,478,723 and 6,312,917, the disclosure of each of which is incorporated herein by reference.

The CYP enzyme can be in any suitable form; for example, the enzyme can be present in the form of microsomes (e.g., from mammalian cells such as liver microsomes) or recombinant CYP microsomes prepared from any eukaryotic or prokaryotic cell such as bacterial, yeast, plant, mammalian or insect cells or can be a reconstituted enzyme, all of which can be obtained from commercial sources (e.g., Invitrogen Corp [PanVera]; Gentest Corp). In representative embodiments, the CYP enzyme is free or essentially free of microsomes. In addition, the enzyme can be purified from a natural source, can be a recombinant enzyme, and/or can be partially or wholly synthetic.

One advantage of recombinant or reconstituted CYP enzyme is that it typically is free or essentially free of detectable catalase, peroxidase and/or superoxide dismutase activity and/or other enzyme activities that can interfere with the methods of the invention by reducing the accumulation of ROS. By “essentially free of,” it is meant that only insignificant activity is present (e.g., less than about 15%, 10%, 7%, 5%, 4%, 3%, 1% or less of the contaminating enzyme activity(ies) that would typically be found in human liver microsome preparations). Methods of assessing the activities of catalase, peroxidase and superoxide dismutase are well known in the art. In particular embodiments, no detectable catalase, peroxidase and/or superoxide dismutase activity is present in the reconstituted CYP enzyme.

An exemplary reconstituted CYP enzyme reagent comprises, consists essentially of or consists of an isolated (e.g., recombinant) CYP enzyme reconstituted in a mixture of purified lipids, NADPH-cytochrome P450 reductase and, optionally, cytochrome b5. Reconstituted CYP enzymes are available from commercial sources (e.g., Invitrogen [Panvera]). In particular embodiments, an “isolated” CYP enzyme is a CYP enzyme that is at least about 25%, 50%, 60%, 70%, 75%, 80%, 85%, 90%, 95%, 97%, 98%, 99% or more pure (w/w).

Suitable conditions for CYP enzyme activity are well known in the art. For example, in particular embodiments, the pH of the reaction mixture is from about pH 6, 6.5 or 7 to about 7.8, 8 or 8.5 and at a temperature from about 18° C. to about 40° C.

Optionally, the enzyme reaction is quenched prior to initiating the detection reaction. Substances that quench enzyme activity are known in the art. In some instances, the detection reagent itself can quench the enzyme reaction. A quenching agent is typically selected that does not destabilize to a significant extent the ROS produced as a side product of the enzyme reaction. Further, the quenching agent is selected so as not to unduly interfere with the detection reaction. The quenching agent can also be selected to be miscible in the detection reagent. One exemplary quenching agent for use with nonaqueous detection reagents is acetonitrile.

The ROS is formed as a side product associated with the primary metabolic activity of the enzyme. ROS are produced as side products of the metabolic activity of, among other enzymes, CYP enzymes and xanthine oxidase. Exemplary ROS include but are not limited to superoxide anion (—O₂⁻), hydrogen peroxide (H₂O₂) and hydroxyl radical (OH—).

The rates of formation of the ROS are proportional to the substrate turnover (i.e., metabolism of the compound by the enzyme) to allow quantitative analysis of the susceptibility of the candidate compound to metabolism by the enzyme. Thus, the production of ROS in the side reaction is proportional to the primary metabolic activity of the enzyme. Further, the formation of the indicator compound is proportional to the production of ROS. Accordingly, formation of the indicator compound is proportional to, and indicative of, the metabolism of the compound by the enzyme.

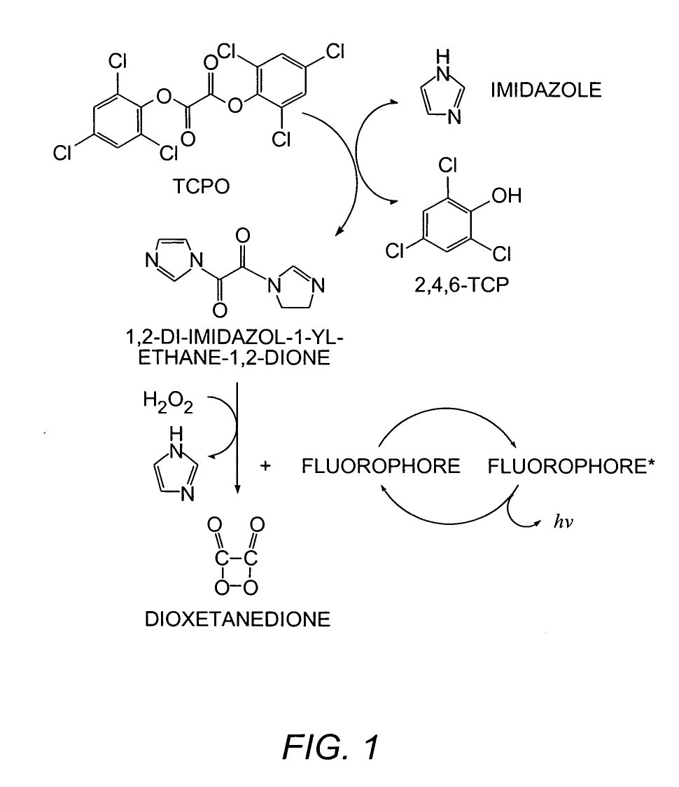

According to the methods of the invention, a portion or all of the enzyme reaction product mixture is contacted with a detection reagent that comprises an indicator compound precursor. In representative embodiments, the detection reagent further comprises a compound that is reactive toward the ROS. Reaction between the reactive compound and the ROS results in the indicator compound being formed. In particular embodiments, the indicator compound precursor is the reactive compound, i.e., the indicator compound reacts directly with the ROS. In other embodiments, the ROS does not react directly with the indicator compound precursor to produce the indicator compound, i.e., there are one or more intermediate chemical reactions between reaction of the ROS and the reactive compound and the conversion of the indicator compound precursor to the indicator compound. Further, in some embodiments the reactive compound that reacts with the ROS can be added directly to the detection reagent; alternatively, it can be formed in the detection reagent from precursor molecules (see, e.g., FIG. 1).

In representative embodiments, only a fraction of the ROS formed as a side product of the metabolism of the compound by the enzyme reacts with the reactive compound to drive production of the indicator compound. According to this embodiment, only a relatively small amount of the ROS produced in the side reaction is diverted to the detection reaction, the amount diverted being insufficient to significantly perturb or alter the rate of metabolism of the compound by the enzyme (i.e., is insufficient to significantly perturb or alter the primary metabolic activity of the enzyme).

In other embodiments, all or substantially all (e.g., at least about 70%, 75%, 80%, 90%, 95%, 98% or more) of the ROS reacts with the reactive compound resulting in production of the indicator compound.

One particular detection system is a peroxyoxalate chemiluminescence system that involves the oxidation of an aryl oxalate ester by the ROS (e.g., hydrogen peroxide) in the presence of an indicator compound precursor (e.g., a fluorophore which is a chemiluminescence compound precursor). The reaction proceeds through a chemically initiated electron exchange luminescence mechanism via a high-energy dioxetandione intermediate. The dioxetandione intermediate forms a charged complex with the fluorophore (e.g., rubrene), which donates one electron to the dioxetandione intermediate. The electron is transferred back to the fluorophore raising it to an excited state with the liberation of light upon relaxation to the ground state.

In representative embodiments the detection reagent comprises trichlorophenyloxalate (TCPO), which can be converted to the aryl oxalate ester substrate of the peroxyoxalate chemiluminescence reaction described in the preceding paragraph. FIG. 1 shows an exemplary reaction scheme in which TCPO in the presence of imidazole and 2,4,6,-TCP (trichlorophenol) is converted to an aryl oxalate ester (1,2-di-imidazol-1-yl-ethane-1,2-dione), which in turn is reactive with hydrogen peroxide to produce a high-energy dioxetandione intermediate. A fluorophore (e.g., rubrene) donates an electron to the dioxetandione intermediate. The electron is then transferred back to the fluorophore raising it to an excited state with the emission of light upon relaxation to the ground state. In this scheme, the aryl oxalate ester is the compound that is reactive toward the ROS, the unexcited fluorophore is the indicator compound precursor, and the excited fluorophore is the indicator compound. The chemiluminescence reaction is typically carried out under non-aqueous conditions because many fluorophores are nonpolar. As described above, suitable nonaqueous solvents include ethyl acetate and methylene chloride.

The “indicator compound” is a compound that can be detected, typically using standard detection techniques, such as fluorescence or chemiluminescence spectrophotometry, colorimetry, and the like. Particular embodiments of the invention use chemiluminescence methods to detect the indicator compound.

Exemplary indicator compound precursors thus include, but are not limited to, compounds that are converted to fluorescent compounds, chemiluminescence compounds, colorimetric indicator compounds, and combinations thereof. Examples of chemiluminescence compound precursors include but are not limited to the fluorophores rubrene, diphenyl anthracine, rhodamine 6G, and rhodamine 123, which can be elevated to an excited state in an electron exchange reaction (as described above).

The indicator compound precursor can be converted directly to the indicator compound or, alternatively, there can be one or more intermediate steps. For example, the immediate precursor of the indicator compound can be generated during the detection reaction.

The methods of the invention further comprise detecting the presence or absence of the indicator compound. The formation of the indicator compound from the indicator compound precursor is proportional to, and indicative of, the production of ROS and the metabolism of the compound by the enzyme. Thus, if the compound is only metabolized at a low rate by the enzyme, less signal from the indicator compound is produced during a set time period as compared with the signal from the indicator compound produced with a compound that is rapidly metabolized by the enzyme. The absence of signal from the indicator compound indicates that the compound was not metabolized by the enzyme or was only metabolized at a level below the detection limits of the assay. The presence of signal from the indicator compound indicates that the compound is susceptible to metabolism by the enzyme. Semi-quantitative or quantitative methods can be used to assess the level, and optionally the rate of metabolism of the compound by the enzyme.

The indicator compound can be detected directly from the detection reaction mixture. Alternatively, a portion of the detection reaction can be removed and the presence of the indicator compound detected therein. For example, an aliquot of the detection reaction can be removed, placed in a multi-well plate, and the presence of the indicator compound determined therefrom. Further, according to this method, multiple aliquots can be removed over time to follow the production of indicator compound (e.g., to determine the rate of metabolism of the compound).

Any compound can be evaluated for susceptibility to metabolism by a enzyme. Further, it is not necessary that the compound be identified or characterized. The term “compound” as used herein is intended to be interpreted broadly and encompasses organic and inorganic molecules. Organic compounds include, but are not limited to polypeptides, lipids, carbohydrates, coenzymes, purines, pyrimidines, and nucleic acid molecules. Exemplary compounds include xenobiotics such as drugs and other therapeutic agents, carcinogens and environmental pollutants, as well as endobiotics such as steroids, fatty acids and prostaglandins.

In particular embodiments, the compound is a candidate compound that is being evaluated as part of a drug screening/identification program. For example, in particular embodiments, the compound is one that has been preliminarily identified as of interest, e.g., because of its therapeutic or potentially therapeutic activity. The compound can be further screened for metabolic stability using the methods of the present invention. Further, a number of different candidate compounds can be screened in parallel using the inventive methods.

In addition, compounds can be obtained from a wide variety of sources including libraries of synthetic or natural compounds. For example, numerous means are available for random and directed synthesis of a wide variety of organic compounds and biomolecules (e.g., using combinatorial chemistry methods). Alternatively, libraries of natural compounds in the form of insect, bacterial, fungal, plant and animal extracts are available or readily produced. Additionally, natural or synthetically produced libraries and compounds are readily modified through conventional chemical, physical and biochemical means. Known pharmacological agents can be subjected to directed or random chemical modifications, such as acylation, alkylation, esterification, amidification to produce structural analogs.

The methods of the present invention can be performed in a cell-free reaction or in a cell-based in vitro reaction. Exemplary cell-based in vitro platforms suitable for modification in accordance with the method of the present invention are described in Parkinson, (1996) Toxicol. Pathol. 24:45-57.

As another aspect, the present invention provides kits for carrying out the inventive methods described above. As one representative embodiment, the invention provides a kit for determining the susceptibility of a compound to metabolism by an enzyme, the kit comprising: (a) an enzyme that produces a ROS as a side product (as described above); (b) trichlorophenyloxalate (TCPO); and (c) a chemiluminescence compound precursor (e.g., a fluorophore such as rubrene). The enzyme can comprise one or more CYP enzymes, as described above. Additionally, the kit can further comprise other components, such as cofactors (e.g., NADP+), buffers, salts, detergents and the like for carrying out the inventive methods. The components of the kit are generally packaged together in a common container, typically including instructions for performing selected embodiments of the methods disclosed herein.

The invention will now be illustrated with reference to certain examples which are included herein for the purposes of illustration only, and which are not intended to be limiting of the invention.

The following abbreviations are used in the Examples: The term “CYP” means cytochrome P450, the term “CYP2D6” means cytochrome P450 2D6, the term “ROS” means reactive oxygen species, the term “TCPO” means trichlorophenyloxalate, the term “CL” means chemiluminescence, the term “LC-MS” means liquid chromatography-mass spectrometry, the term “HPLC” means high performance liquid chromatography, and the term “DMSO” means “dimethyl sulfoxide.”

### EXAMPLES

### Development of a High-Throughput Chemiluminescence-|Based Cytochrome P450 (CYP) Metabolic Stability Assay

1. Materials and Methods

1.1. Materials. Purified recombinant CYP2D6 was purchased from Invitrogen Corp. (PanVera) in a catalytically active reconstituted format (RECO™ System). Recombinant CYP2D6 microsomes were purchased from BD Biosciences (GenTest™). All other supplies were purchased from Sigma-Aldrich Chemicals (St. Louis, Mo.). The buffer used in all reactions was 100 mM potassium phosphate buffer (pH 7.4) containing 3.3 mM MgCl₂ and is referred to as “buffer” in the following sections.

1.2. Chemiluminescence Metabolic Stability Assay.

1.2.1. CYP2D6 Reactions.

Stock solutions of NADPH were freshly prepared before each experiment in buffer. Test compounds were prepared as 20 mM stock solutions in DMSO and diluted to 2 mM in acetonitrile. The final acetonitrile concentration was the same among all samples and did not exceed 1% (v/v). Unless otherwise specified, reaction mixtures consisted of 20 μM substrate, 6.65 pmol CYP2D6 (in the form of a reconstituted RECO® system) and 1 mM NADPH prepared in buffer to a final volume of 200 μl. Reactions were performed at 37° C. in 500 μL deep 96-well blocks. Reactions were incubated for 3 min prior to adding NADPH. Aliquots of the reaction mixture (10 μL) were removed at 0 (immediately after the addition of NADPH), 5,10, 20 and 30 min and quenched with 20 μL of acetonitrile and placed in 96-well polypropylene plates. ROS formation was quantified using a chemiluminescence reaction as described below.

1.2.2. Chemiluminescence Reactions.

Rubrene, TCPO and imidazole were prepared in ethyl acetate as 0.5, 5.0 and 4.5 mM stocks, stored in amber vials and used within 48 hr. A 10:1:1 mixture of rubrene:TCPO:imidazole (CL mix) was prepared just before each experiment. A 150 μL aliquot of CL mix was dispensed into 96-well plates containing the quenched CYP reaction (prepared as described in Section 1.2.1 above) and chemiluminescence was measured immediately thereafter at 1 min intervals for a total of 10 min using a Molecular Devices Microplate Reader.

1.3. Metabolic Stability by Parent Compound Loss Using LC-MS.

1.3.1. CYP2D6 Reactions.

Stock solutions of NADPH were freshly prepared before each experiment in buffer. Test compounds were prepared as 20 mM stock solutions in DMSO and diluted to 2 mM in acetonitrile. The final acetonitrile concentration was the same among all samples and did not exceed 1% (v/v). Unless otherwise specified, reaction mixtures consisted of 5 μM substrate, 30 pmol/mL recombinant CYP2D6 microsomes (GenTest™) and 1 mM NADPH prepared in buffer to a final volume of 750 μl. Reactions were performed at 37° C. in 1.2 mL marsh tubes. Reactions were incubated for 3 min prior to adding NADPH. Aliquots of the reaction mixture (100 μL) were removed at 0 (immediately after the addition of NADPH), 5, 10, 20 and 30 min and quenched with 200 μL of acetonitrile (containing 2.5 μM terfenadine). Samples were placed at −20° C. for 1 hr and centrifuged using a Thermo IEC Centra GP8R table top centrifuge (3,000 rpm for 10 min). The supernatant fraction was transferred to 500 μL 96-well blocks for LC-MS analysis.

1.3.2. LC-MS Analysis.

LC-MS analysis was performed using a HPLC system consisting of a Shimadzu LC-10AD pump and Perkin Elmer Series 200 Autosampler coupled to an Applied Biosystems Sciex API 3000 triple-quadrapole mass spectrometer. Test compounds were separated on an Agilent ZORBAX® Bonus-RP column (30×2.1 mm, 3.5 μm particle size) maintained at 30° C. Three μL of sample was injected onto the column equilibrated with 100% buffer A (10% acetonitrile, 8.75 mM formic acid, 3.75 mM ammonium formate). Buffer A was held constant for the first 0.5 min of the run followed by a ramp at 0.6 min to 100% buffer B (80% acetonitrile, 8.75 mM formic acid, 3.75 mM ammonium formate) and held constant for 2 min. The column was equilibrated with 100% buffer A for 2 min between each run. The HPLC eluant was directly introduced into the mass spectrometer equipped with a Turbo Ionspray interface operated at 4500 V in the positive mode. Nitrogen was used as the nebulizer gas at 8 L/min and as the drying gas heated to 425° C. at a flow rate of 7 L/min to evaporate solvents in the spray chamber. Compounds were detected by single ion monitoring using the following parameters: declustering potential, 35 V; focusing potential, 200 V; exit potential, 10 V.

2. Results.

2.1. Optimization of stop solution and chemiluminescent components. The addition of a chemiluminescence (CL) mixture containing rubrene/TCPO/imidazole to phosphate buffer resulted in a low level of CL production. H₂O₂ added to phosphate buffer prior to the addition of the CL mix resulted in a concentration dependent increase in CL. The amount of rubrene, TCPO and imidazole in the CL mix was adjusted so as to give the maximum amount of CL signal. Organic solvents had a significant effect on H₂O₂-dependent CL production and thus have a significant effect on quantifying H₂O₂ in CYP reactions. The addition of DMSO or isopropyl alcohol to buffer resulted in the complete loss in CL signal over background levels. Acetonitrile appeared to be optimal as it was the only organic solvent tested that did not significantly diminish the CL signal when added to a phosphate buffer- H₂O₂ solution. However, the volume of acetonitrile added to a phosphate buffer- H₂O₂ solution had a significant effect on the intensity of the CL signal and the time that it took to reach its maximum value after adding the CL mix. A 1:2 ratio of CYP reaction:acetonitrile (10 μLf CYP added to 20 μL of acetonitrile) was the optimum condition for quenching the CYP reactions.

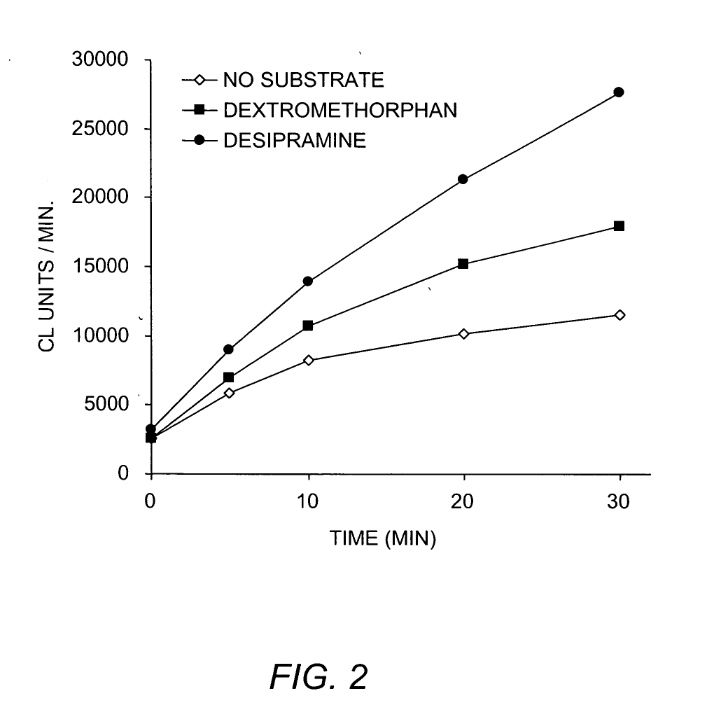

2.2. ROS-dependent CL formation in CYP reactions. The production of ROS in CYP reactions was quantified using the conditions as described above. Aliquots of a CYP2D6 reaction were removed at several time intervals after the addition of NADPH and the ROS quantified as presented in FIG. 2. The no substrate samples showed a moderate level of CL production over the 30 min time period of the experiment. The amount of ROS production increased rapidly over the first 10 min of the experiment and thereafter decreased to about one-half the initial rate for the following 20 min. The addition of 20 μM dextromethorphan (a high-turnover CYP2D6 substrate) to the CYP reactions resulted in an approximately two-fold increase in the rate of CL production, indicating an increase in the amount of ROS in the presence of a rapidly metabolized substrate. Furthermore, desipramine, which is also a rapidly metabolized CYP2D6 substrate, resulted in approximately 2-fold higher rate of ROS-dependent CL production in comparison to dextromethorphan and an approximately 4-fold higher rate over background (no substrate). Thus, it was established that the ROS-dependent CL assay was able to detect the ROS produced in CYP reactions and that the addition of a highly metabolized substrate (dextromethorphan and desipramine) to these reactions resulted in an increase in signal 2 to 4-fold over background levels.

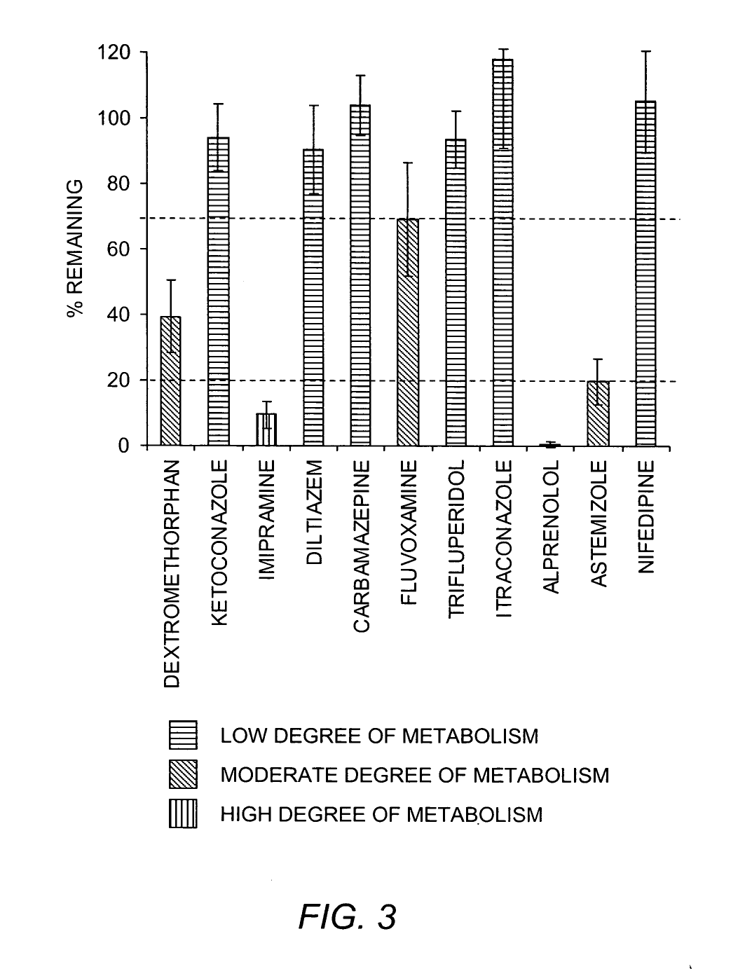

2.3. The Ability of the ROS-Based CL Reaction to Measure Metabolic Stability. Eleven compounds were selected to test whether the ROS-based CL reaction could predict their relative metabolic stability towards CYP2D6 oxidative metabolism. Compounds were first rank ordered based on their metabolic susceptibility towards CYP2D6 by measuring parent compound loss using LC-MS. Compounds were placed into three categories based on the following criteria: (1) low (more than 75% of parent compound remaining after 10 min), (2) moderate (between 20 and 75% of parent compound remaining after 10 min), and (3) high (less than 20% of parent compound remaining after 10 min). Of the 11 compounds tested (FIG. 3); two compounds were identified as highly metabolized, three compounds were identified as moderately metabolized, and six compounds were identified as having low metabolism.

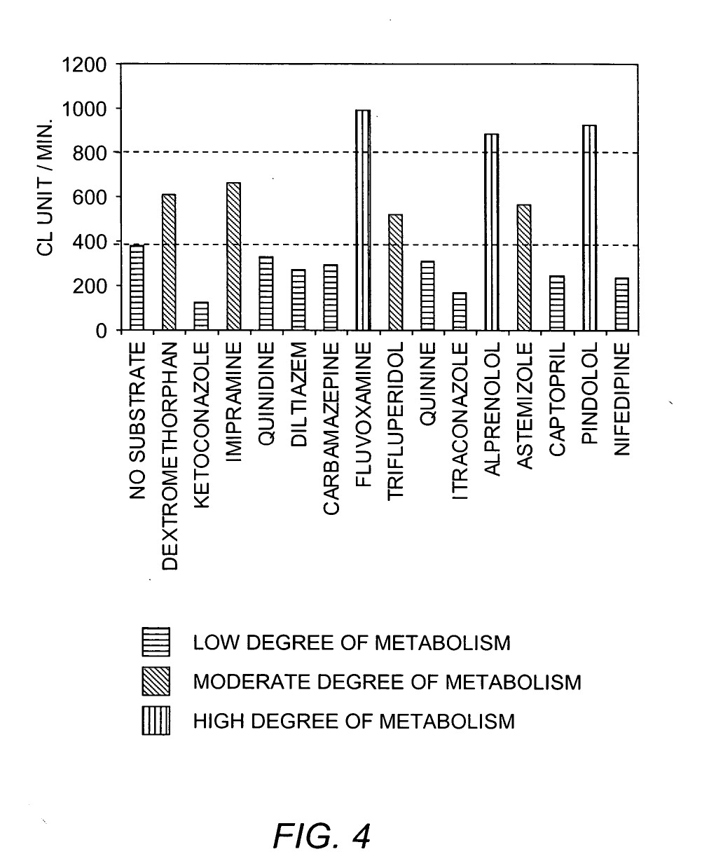

Fifteen compounds, including 11 of the compounds described above, were tested using the ROS-based CL reaction and the results are presented in FIG. 4. The compounds were rank ordered for their metabolic susceptibility based on the following criteria; (1) low (a rate of CL production equal to or below that observed in the absence of substrate (400 Units/min)), (2) moderate (a rate of CL production between 400 (background) and 800 CL Units/min), and (3) high (a rate of CL production above 800 Units/min). Nine of the compounds were characterized as having low metabolism based on the ROS-based CL reaction, four as having moderate metabolism, and three were ranked as being highly metabolized.

A comparison of the two methods for rank ordering compounds based on their metabolic stability towards CYP2D6 metabolism is presented in Table 1. Eight out of the eleven compounds were rank ordered the same using either approach. Of the three compounds rank ordered differently, two of these compounds (imipramine and fluvoxamine) were identified as being metabolized using this approach, but with a rank order that different by 1. Thus, these results indicate that the ROS-based CL reaction is a useful screen for identifying compounds susceptible to CYP2D6 metabolism that does not rely upon more arduous, time-consuming and expensive LC-MS analysis.

TABLE 1
Rank ordering of compounds based on their metabolic stability
towards CYP2D6 oxidative metabolism using the ROS-based CL
reaction vs. measuring parent compound loss by LC-MS.

| Compound | CL rank order** | LC-MS rank order |
|---|---|---|
| no substrate | 1 | — |
| dextromethorphan | 2 | 2 |
| ketoconazole | 1 | 1 |
| imipramine | 2 | 3 |
| diltiazem | 1 | 1 |
| carbamazepine | 1 | 1 |
| fluvoxamine | 3 | 2 |
| trifluperidol | 2 | 1 |
| itraconazole | 1 | 1 |
| alprenolol | 3 | 3 |
| astemizole | 2 | 2 |
| nifedipine | 1 | 1 |

**A rank of 1 = low degree of metabolism; 2 = moderate degree of metabolism; 3 = high degree of metabolism.

Although the foregoing invention has been described in some detail by way of illustration and example for purposes of clarity of understanding, it will be obvious that certain changes and modifications may be practiced within the scope of the appended claims.

That which is claimed is:

1. A method of determining the susceptibility of a compound to metabolism by an enzyme, the method comprising:
(a) contacting a compound with an enzyme to produce a reaction product mixture, wherein metabolism of the compound by the enzyme produces a reactive oxygen species as a side product in the reaction product mixture; then
(b) contacting at least some of the reaction product mixture of (a) with a detection reagent comprising an indicator compound precursor, wherein reaction between reactive oxygen species in the reaction product mixture and the detection reagent results in the production of an indicator compound from the indicator compound precursor; and
(c) detecting the presence or absence of the indicator compound, wherein the presence of the indicator compound indicates the susceptibility of the compound to metabolism by the enzyme, thereby determining the susceptibility of a compound to metabolism by the enzyme.

2. The method of claim 1, wherein the enzyme is a cytochrome P450 enzyme.

3. The method of claim 2, wherein the cytochrome P450 enzyme is selected from the group consisting of CYP1A2, CYP2C9, CYP2C10, CYP2C19, CYP2D6, CYP3A4, CYP2E, and a combination thereof.

4. The method of claim 2, wherein the cytochrome P450 enzyme is a human cytochrome P450 enzyme.

5. The method of claim 2, wherein the cytochrome P450 enzyme is derived from an animal or plant.

6. The method of claim 2, wherein the cytochrome P450 enzyme is a recombinant enzyme.

7. The method of claim 2, wherein the cytochrome P450 enzyme is a reconstituted cytochrome P450 enzyme.

8. The method of claim 7, wherein the reconstituted cytochrome P450 enzyme comprises an isolated cytochrome P450 enzyme and NADPH-cytochrome P450 reductase.

9. The method of claim 7, wherein the reconstituted cytochrome P450 enzyme is essentially free of detectable catalase, peroxidase and superoxide dismutase activity.

10. The method of claim 1, wherein the reaction of (a) is carried out in an aqueous phase and the reaction of (b) is carried out in a nonaqueous phase.

11. The method of claim 1, wherein at least some of the reaction product mixture of (a) is removed at two or more time points and contacted with the detection reagent.

12. The method of claim 1, wherein the reactive oxygen species does not react directly with the indicator compound precursor to produce the indicator compound.

13. The method of claim 1, wherein the reactive oxygen species is selected from the group consisting of hydrogen peroxide, superoxide anion, hydroxyl radical, and a combination thereof.

14. The method of claim 1, wherein the indicator compound precursor is selected from the group consisting of a fluorogenic compound precursor, a calorimetric compound precursor, a chemiluminescent compound precursor, and a combination thereof.

15. The method of claim 14, wherein the indicator compound precursor is a chemiluminescent compound precursor.

16. The method of claim 1, wherein the detection reagent comprises trichlorophenyloxalate (TCPO).

17. The method of claim 16, wherein the indicator compound precursor is rubrene.

18. The method of claim 1, wherein the method is qualitative.

19. The method of claim 1, wherein the method is quantitative.

20. The method of claim 19, further comprising determining the production of reactive oxygen species over a period of time to thereby determine a rate of metabolism of the compound by the enzyme.

21. The method of claim 1, wherein the reactions of (a) and (b) are carried out in a multi-well plate.

22. The method of claim 21, wherein the method is carried out on a plurality of compounds in parallel.

23. A method of determining the susceptibility of a compound to metabolism by an enzyme, the method comprising:
(a) contacting a compound with an enzyme to produce a reaction product mixture, wherein metabolism of the compound by the enzyme produces a reactive oxygen species as a side product in the reaction product mixture;
(b) removing at least some of the reaction product mixture of (a) and contacting with a detection reagent comprising an indicator compound precursor, wherein reaction between reactive oxygen species in the reaction product mixture and the detection reagent results in the production of an indicator compound from the indicator compound precursor; and
(c) detecting the presence or absence of the indicator compound, wherein the presence of the indicator compound indicates the susceptibility of the compound to metabolism by the enzyme, thereby determining the susceptibility of a compound to metabolism by the enzyme.

24. The method of claim 23, wherein the enzyme is a cytochrome P450 enzyme.

25. The method of claim 24, wherein the cytochrome P450 enzyme is selected from the group consisting of CYP1A2, CYP2C9, CYP2C10, CYP2C19, CYP2D6, CYP3A4, CYP2E, and a combination thereof.

26. The method of claim 24, wherein the cytochrome P450 enzyme is a reconstituted cytochrome P450 enzyme.

27. The method of claim 23, wherein at least some of the reaction product mixture of (a) is removed and is added to a separate reaction vessel to carry out the reaction of (b).

28. The method of claim 23, wherein the reaction of (a) is carried out in an aqueous phase and the reaction of (b) is carried out in a nonaqueous phase.

29. The method of claim 23, wherein the reactive oxygen species does not react directly with the indicator compound precursor to produce the indicator compound.

30. The method of claim 23, wherein the reactive oxygen species is selected from the group consisting of hydrogen peroxide, superoxide anion, hydroxyl radical, and a combination thereof.

31. The method of claim 23, wherein the indicator compound precursor is selected from the group consisting of a fluorogenic compound precursor, a colorimetric compound precursor, a chemiluminescent compound precursor, and a combination thereof.

32. The method of claim 23, wherein the detection reagent comprises trichlorophenyloxalate (TCPO).

33. The method of claim 32, wherein the indicator compound precursor is rubrene.

34. A method of determining the susceptibility of a compound to metabolism by a cytochrome P450 enzyme, the method comprising:
(a) contacting a compound with a cytochrome P450 enzyme to produce a reaction product mixture, wherein metabolism of the compound by the cytochrome P450 enzyme produces a reactive oxygen species as a side product in the reaction product mixture;
(b) contacting all or a portion of the reaction product mixture of (a) with a detection reagent comprising a chemiluminescent compound precursor and trichlorophenyloxalate (TCPO), wherein reaction between reactive oxygen species in the reaction product mixture and the detection reagent results in the production of a chemiluminescent compound from the chemiluminescent compound precursor; and
(c) detecting the presence or absence of the chemiluminescent compound, wherein the presence of the chemiluminescent compound indicates the susceptibility of the compound to metabolism by the cytochrome P450 enzyme, thereby determining the susceptibility of a compound to metabolism by the cytochrome P450 enzyme.

35. The method of claim 34, wherein the cytochrome P450 enzyme is selected from the group consisting of CYP1A2, CYP2C9, CYP2C10, CYP2C19, CYP2D6, CYP3A4, CYP2E, and a combination thereof.

36. The method of claim 34, wherein the cytochrome P450 enzyme is a human cytochrome P450 enzyme.

37. The method of claim 34, wherein the cytochrome P450 enzyme is a reconstituted cytochrome P450 enzyme.

38. The method of claim 37, wherein the reconstituted cytochrome P450 enzyme comprises an isolated cytochrome P450 enzyme and NADPH-cytochrome P450 reductase.

39. The method of claim 37, wherein the reconstituted cytochrome P450 enzyme is essentially free of detectable catalase, peroxidase and superoxide dismutase activity.

40. The method of claim 34, wherein (a) and (b) are carried out as separate reactions.

41. The method of claim 40, wherein the reaction of (a) is carried out in an aqueous phase and the reaction of (b) is carried out in a nonaqueous phase.

42. The method of claim 40, wherein at least some of the reaction product mixture of (a) is removed and is added to a separate reaction vessel to carry out the reaction of (b).

43. The method of claim 34, wherein at least some of the reaction product mixture of (a) is removed at two or more time points and contacted with the detection reagent.

44. The method of claim 34, wherein the reactive oxygen species is selected from the group consisting of hydrogen peroxide, superoxide anion, hydroxyl radical, and a combination thereof.

45. The method of claim 34, wherein the chemiluminescent compound precursor is rubrene.

46. The method of claim 34, wherein the method is qualitative.

47. The method of claim 34, wherein the method is quantitative.

48. The method of claim 47, further comprising determining the production of reactive oxygen species over a period of time to thereby determine a rate of metabolism of the compound by the cytochrome P450 enzyme.

49. The method of claim 34, wherein the reactions of (a) and (b) are carried out in a multi-well plate.

50. The method of claim 49, wherein the method is carried out on a plurality of compounds in parallel.

51. A kit for determining the susceptibility of a compound to metabolism by an enzyme, the kit comprising:
(a) an enzyme;
(b) trichlorophenyloxalate (TCPO); and
(c) a chemiluminescent compound precursor.

52. The kit of claim 51, wherein the enzyme is a cytochrome P450 enzyme.

53. The kit of claim 52, wherein the kit comprises two or more cytochrome P450 enzymes.

54. The kit of claim 52, wherein the cytochrome P450 enzyme is a reconstituted cytochrome P450 enzyme.

55. The kit of claim 54, wherein the reconstituted cytochrome P450 enzyme comprises an isolated cytochrome P450 enzyme and NADPH-cytochrome P450 reductase.

56. The kit of claim 54, wherein the reconstituted cytochrome P450 enzyme is essentially free of detectable catalase, peroxidase and superoxide dismutase activity.

57. The kit of claim 52, wherein the cytochrome P450 enzyme is selected from the group consisting of CYP1A2, CYP2C9, CYP2C10, CYP2C19, CYP2D6, CYP3A4, CYP2E, and a combination thereof.

58. The kit of claim 52, wherein the cytochrome P450 enzyme is a human cytochrome P450 enzyme.

59. The kit of claim 51, wherein the chemiluminescent compound precursor is rubrene.
